(19) United States
(12) Patent Application Publication
(10) Pub. No.: US 2024/0382152 A1
(43) Pub. Date: Nov. 21, 2024

(54) SLEEP STATE MONITORING EARPLUG

(71) Applicant: SHUNSIN TECHNOLOGY (ZHONG SHAN) LIMITED, Zhongshan (CN)

(72) Inventor: HONG-GUANG HUANG, Shenzhen (CN)

(21) Appl. No.: 18/423,212

(22) Filed: Jan. 25, 2024

(30) Foreign Application Priority Data
May 16, 2023 (CN) 202310555887.X

(51) Int. Cl. A61B 5/00; A61B 5/06; A61B 5/08; A61B 5/1455

(52) U.S. Cl. CPC A61B 5/4809; A61B 5/067; A61B 5/0816; A61B 5/14551; A61B 5/6817; A61B 5/7405; A61B 5/746; A61B 2562/0219

(57) ABSTRACT

A sleep state monitoring earplug designed for insertion into the ear canal. The earplug comprises a detection module, which includes a processing unit, a respiration detection unit, and an alert unit. The respiration detection unit generates respiratory rate information to the processing unit. The processing unit assesses the respiratory rate information and triggers an alert signal to the alert unit when the respiratory rate below a respiratory threshold, causing the alert unit emits a warning signal to awaken users experiencing respiratory cessation.

### FIELD

The subject matter herein generally relates to a physiological state detection device, in particular to a sleep state monitoring earplug designed to awaken individuals suffering from sleep apnea.

### BACKGROUND

The main symptom of sleep apnea is the occurrence of breathing cessation or reduced breathing during sleep due to the collapse of the upper airway. As patients are in a state of sleep when these symptoms occur, the patients often cannot self detect and may require examinations or reminders from bed partners. Failure to timely awaken the patient when sleep apnea occurs may lead to unfortunate consequences.

Currently, detections of sleep apnea require users wearing monitoring devices during sleep to detect physiological data such as breathing, heart rate, and blood oxygen levels. However, existing devices for detecting sleep apnea are relatively bulky and uncomfortable to wear which may significantly impacting the sleep quality of patients during testing. Additionally, these devices may adversely affect the accuracy of physiological data collection, leading to less precise detection results.

Designing a sleep state monitoring earplug that is lightweight, comfortable to wear, and capable of detecting physiological data would effectively address the issues mentioned above.

### BRIEF DESCRIPTION OF THE DRAWINGS

Many aspects of the present disclosure are better understood with reference to the following drawings. The components in the drawings are not necessarily drawn to scale, the emphasis instead being placed upon clearly illustrating the principles of the present disclosure. It will be appreciated that for simplicity and clarity of illustration, where appropriate, reference numerals have been repeated among the different figures to indicate corresponding or analogous elements.

FIG. 1 is a system block diagram of a first embodiment of the present disclosure;

FIG. 2 is a method flowchart of implementing the first embodiment of the present disclosure;

FIG. 3 is a system block diagram of a second embodiment of the present disclosure;

FIG. 4 is a method flowchart implementing the second embodiment of the present disclosure;

FIG. 5 is a method flowchart of a third embodiment of the disclosure.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean “at least one”.

The term “coupled” is defined as connected, whether directly or indirectly through intervening components, and is not necessarily limited to physical connections. The connection can be such that the objects are permanently connected or releasably connected. The term “comprising,” when utilized, means “including, but not necessarily limited to;” it specifically indicates open-ended inclusion or membership in the so-described combination, group, series, and the like.

The sleep state monitoring earplug 1 of the first embodiment of the present invention can be inserted into the user's ear canal before bedtime. In addition to serving as noise-cancelling earplugs, the sleep state monitoring earplug 1 can be used to detect the user's physiological state. It is capable of determining whether the user is experiencing sleep apnea. When sleep apnea is detected, the earplug can actively awaken the user, preventing potential risks to the user's life caused by oxygen deficiency.

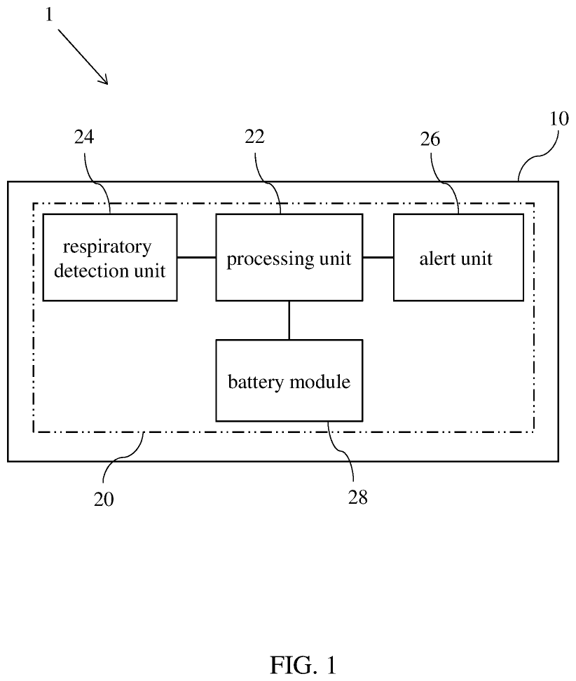

Referencing FIG. 1, the sleep state monitoring earplug 1 includes a main body 10 and a detection module 20, where the detection module 20 is positioned within the main body 10. The main body 10 can be made of materials such as foam, wax, or silicone, and is shaped to conform to the ear canal, such as in a rectangular shape. The detection module 20 comprises a processing unit 22, a respiration detection unit 24, an alert unit 26, and a battery module 28. The respiration detection unit 24, alert unit 26, and battery module 38 are connected to the processing unit 22. The respiration detection unit 24 and the alert unit 26 are controlled by the processing unit 22. The respiration detection unit 24 can generate respiratory rate information to the processing unit 22, enabling the processing unit 22 to generate corresponding signals to control the alert unit 26 based on the respiratory rate information. The battery module 28 provides power to the processing unit 22, respiration detection unit 24, and alert unit 26 for their operation.

In this embodiment, the processing unit 22 can be a device capable of information processing, such as a Central Processing Unit (CPU) or a control chip, to process the information collected by the respiration detection unit 24. The respiration detection unit 24 in this embodiment can be a microphone, such as a dual-microphone noise reduction module. The respiration detection unit 24 includes a first microphone and a second microphone, positioned on opposite sides of the main body 10. When the main body 10 is inserted into the ear canal, the first microphone is within the ear canal, collecting the user's breathing sound, while the second microphone is exposed outside the ear canal, capturing ambient noise. Therefore, the respiration detection unit 24, based on the information detected by the second microphone outside the ear canal, eliminates environmental noise, retaining only the breathing sound detected inside the ear to generate respiratory rate information. The alert unit 26 can be a device capable of emitting sound, such as a speaker. The battery module 28 can be a rechargeable secondary battery capable of wireless charging, such as nickel-cadmium batteries, nickel-hydrogen batteries, lithium-ion batteries, or lithium-polymer batteries that support wireless charging.

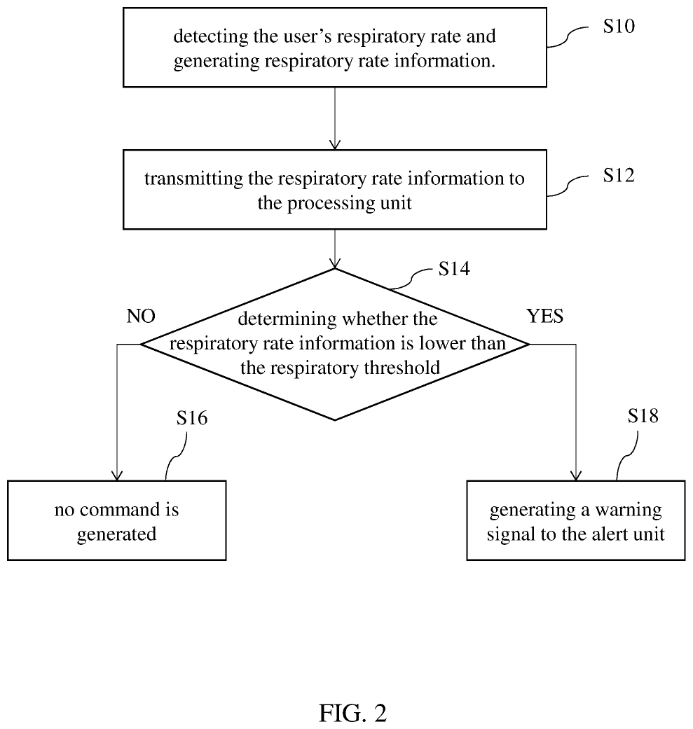

Next, please refer to FIG. 1 and FIG. 2 to explain the method flow of the first embodiment. First, proceed to step S10, where the respiration detection unit 24 detects the user's respiratory rate and generates respiratory rate information. Then, move to step S12, where the respiration detection unit 24 transmits the respiratory rate information to the processing unit 22. In step S14, the processing unit 22 determines whether the respiratory rate information is lower than the respiratory threshold. If NO, proceed to step S16, where no command is generated. If YES, the respiratory rate information is determined to be lower than the respiratory threshold, move to step S18. The processing unit 22 then generates a warning signal to the alert unit 26, causing the alert unit 26 to emit an audible warning based on the warning signal. This audible warning serves to awaken the user, preventing the user from experiencing prolonged oxygen deficiency.

In this embodiment, the respiratory threshold is set at 5 breaths per minute, meaning a breathing rate of 5 breaths per minute. When the breathing rate is below 5 breaths per minute for one minute, it can be determined that breathing is approaching cessation or respiratory weakness, with a high probability of sleep apnea occurrence.

In addition to the above embodiment, the present invention also provides a second embodiment. In this embodiment, the sleep state monitoring earplug 2 still allows the user to insert it into the ear canal before sleep, serving as both a noise-cancelling earplug and a device for detecting the user's physiological state. This allows for the determination of whether the user is experiencing sleep apnea. When sleep apnea is detected, the earplug can actively awaken the user, preventing potential risks to the user's life caused by oxygen deficiency.

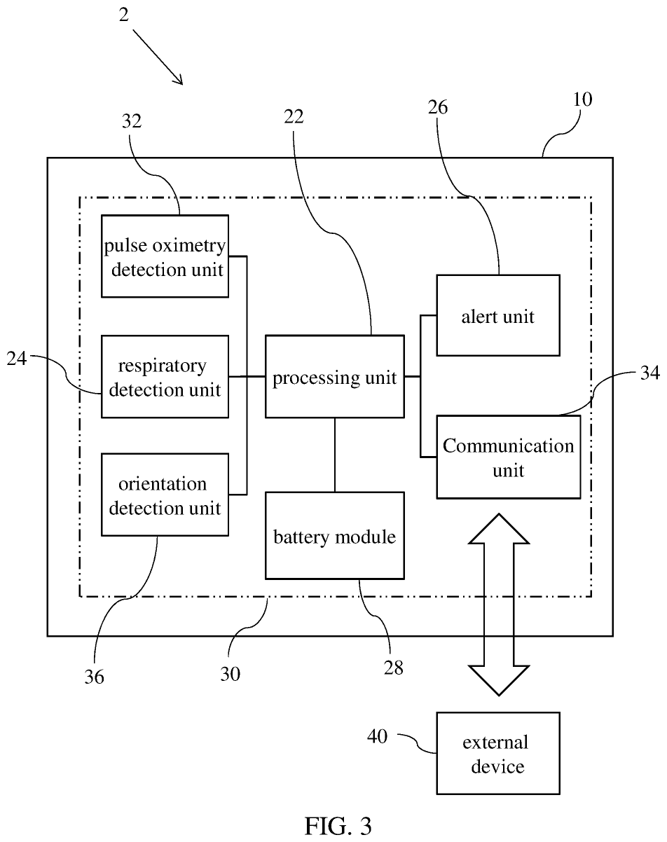

As shown in FIG. 3, the sleep state monitoring earplug 2 also includes a main body 10 and a detection module 30. The main body 10 is the same as in the previous embodiment and can be made of materials such as foam, wax, or silicone, designed to fit the shape of the ear canal, such as a rectangular shape. In this embodiment, the detection module 30 includes the processing unit 22, respiration detection unit 24, alert unit 26, and battery module 28, as in the first embodiment. Additionally, it includes a pulse oximetry detection unit 32, a communication unit 34, and an orientation detection unit 36. The pulse oximetry detection unit 32, communication unit 34, and orientation detection unit 36 are all connected to the processing unit 22 to receive control from the processing unit 22. The connection and information transmission methods of the processing unit 22, respiration detection unit 24, alert unit 26, and battery module 28 are the same as in the previous embodiment and will not be repeated here.

The pulse oximetry detection unit 32 is a pulse oximeter that generates blood oxygen information and pulse rate information for the processing unit 22. In this embodiment, the pulse oximetry detection unit 32 uses a light-emitting element to emit light that is directed into the skin of the ear canal. It then receives the reflected light, measuring changes in light absorption to obtain a Photoplethysmography (PPG) waveform. This allows the unit to obtain blood oxygen information and pulse rate information. The main body 10, when inserted into the ear canal, closely adheres to it, blocking external light and creating a dark environment, which provides an optimal detection environment for Photoplethysmography, enhancing signal stability.

The communication unit 34, in addition to being connected to the processing unit 22, is further connected to at least one external device 40. This enables the communication unit 34 to receive control from the processing unit 22 and transmit blood oxygen information, pulse rate information, and respiratory rate information to the external device 40. In this embodiment, the communication unit 34 may be a wireless communication device providing wireless communication using radio waves, such as Bluetooth devices, wireless network devices, etc. The external device 40 functions as a backend calculator, such as a personal computer, smartphone, tablet, etc.

The orientation detection unit 36 is used to detect angles. In this embodiment, the orientation detection unit 36 is a gyroscope that generates angle information for the processing unit 22.

In this embodiment, the processing unit 22 is the same as in the previous embodiment. When the processing unit 22 determines that the respiratory rate information is below the respiratory threshold, meaning the respiratory rate is less than 5 breaths per minute, the processing unit 22 will generate a warning signal directly to the alert unit 26. The alert unit 26 then issues an audible alert to awaken the user based on the warning signal.

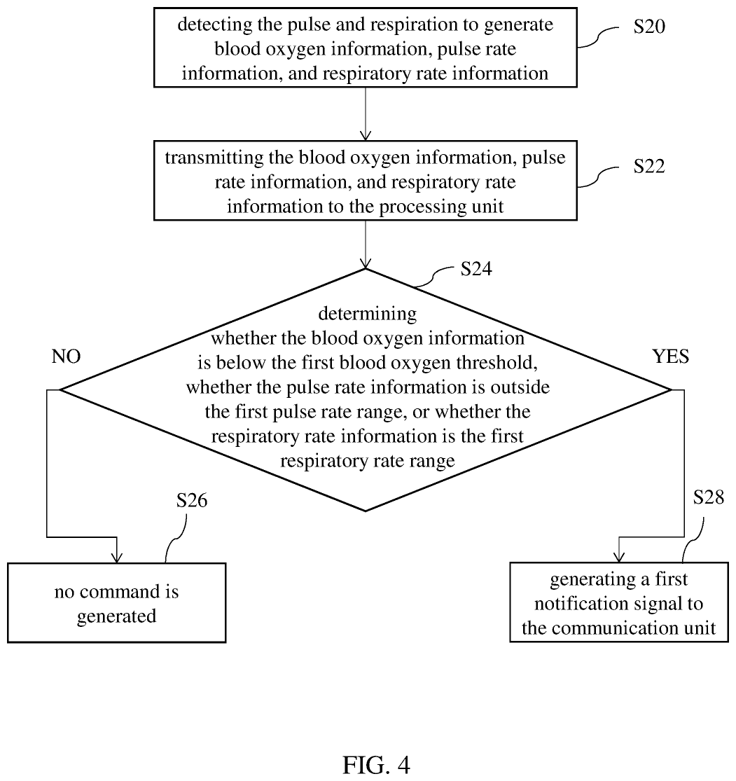

In addition, this embodiment provides another information processing method, please refer to FIG. 3 and FIG. 4 to illustrate the flowchart of the method of the second embodiment of the present invention. First, step S20, where the pulse oximetry detection unit 32 and the respiratory detection unit 24 respectively detect the user's pulse and respiration to generate blood oxygen information, pulse rate information, and respiratory rate information. Next, enter step S22, where the pulse oximetry detection unit 32 and the respiratory detection unit 24 transmit the blood oxygen information, pulse rate information, and respiratory rate information to the processing unit 22. Then, enter step S24, where the processing unit 22 determines whether the blood oxygen information is below the first blood oxygen threshold, whether the pulse rate information is outside (above or below) the first pulse rate range, or whether the respiratory rate information is outside (above or below) the first respiratory rate range. If NO, proceed to step S26 and no command is generated. If YES, indicating that the blood oxygen information is below the first blood oxygen threshold, the pulse rate information is outside the first pulse rate range, or the respiratory rate information is outside the first respiratory rate range, proceed to step S28. The processing unit 22 generates a first notification signal to the communication unit 34, prompting the communication unit 34 to transmit the first notification signal and the detected blood oxygen information, pulse rate information, and respiratory rate information to the external device 40. In this embodiment, the first blood oxygen threshold is set at 92 mm Hg, the first pulse rate range is 50-90 bpm (beats per minute), and the first respiratory rate range is 10-20 breaths per minute. When the blood oxygen information is below 92 mm Hg, the pulse rate information is outside the range of 50-90 bpm, or the respiratory rate information is outside the range of 10-20 breaths per minute, it indicates that the blood oxygen, pulse, or respiratory rate has exceeded the normal range, and physiological signals are in an unstable state. At this time, the first notification signal and the detected blood oxygen information, pulse rate information, and respiratory rate information are transmitted to the external device 40 on the backend.

The first notification signal can alert the backend personnel at the external device 40 to pay attention to the user's sleep condition. If the backend personnel, upon reviewing the blood oxygen information, pulse rate information, and respiratory rate information, determine that the user needs to be awakened, they can issue a warning notification to the communication unit 34. The communication unit 34 then transmits the warning notification to the processing unit 22, enabling the processing unit 22 to control the alert unit 26 to issue a warning to awaken the user. At the same time, the external device 40 can also store this information, such as the time of receiving the message and the blood oxygen information, pulse rate information, and respiratory rate information detected at that moment. This data can be recorded to track the user's sleep status and provide reference for subsequent treatment.

In addition, in this embodiment, the sleep state detection earplug 2 can also adjust the alert level based on the user's sleeping position. For example, if the detection module 30 determines that the user is currently in a supine position, it may raise the alert level. The details are explained as follows.

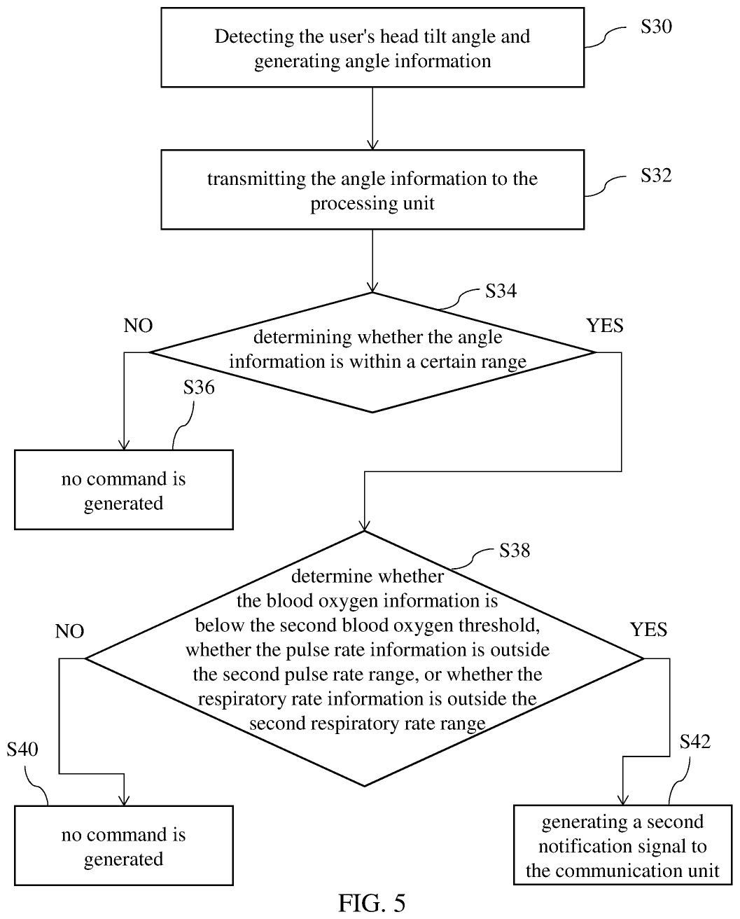

Please refer to FIG. 3 and FIG. 5. First, enter step S30. In this embodiment, the user's head tilt angle is detected by the orientation detection unit 36, generating angle information. Then, enter step S32, the orientation detection unit 36 transmits the angle information to the processing unit 22. In step S34, the processing unit 22 determines whether the angle information is within a certain range. Based on the current head tilt angle of the user, it assesses whether the user is in a supine position. For example, when the processing unit 22 receives an angular velocity on the Z-axis greater than 1, it determines that the user is currently moving the head and adopting a supine position.

If step S34 is determined to be NO, proceed to step S36, and no command is generated. If step S34 is determined to be YES, indicating that the angle information is within the specified range, meaning the user is in a supine position, then proceed to step S38. In step S38, the processing unit 22 will determine blood oxygen information, pulse rate information, or respiratory rate information based on the second blood oxygen threshold, second pulse rate range, or second respiratory rate range. The first blood oxygen threshold is higher than the second blood oxygen threshold. The second pulse rate range is relatively reduced compared to the first pulse rate range, and the second respiratory rate range is relatively reduced compared to the first respiratory rate range. Therefore, it can be understood that the judgment criteria of the processing unit 22 become more stringent.

In detail, in step S38, the processing unit 22 will determine whether the blood oxygen information is below the second blood oxygen threshold, whether the pulse rate information is outside (above or below) the second pulse rate range, or whether the respiratory rate information is outside (above or below) the second respiratory rate range. If NO, proceed to step S40, and no command is generated. However, if step S38 determines to be YES, proceed to step S42, generating a second notification signal to the communication unit 34. The communication unit 34 then transmits the second notification signal to the external device 40. In this embodiment, the second blood oxygen threshold is set at 95 mm Hg, the second pulse rate range is 60-85 bpm, and the second respiratory rate range is 12-18 breaths per minute. In other words, when the blood oxygen information is below 95 mm Hg, the pulse rate information is outside the range of 60-85 bpm, or the respiratory rate information is outside the range of 12-18 breaths per minute, a second notification signal is generated to the communication unit 34, notifying the backend personnel at the external device 40. At the same time, the detected blood oxygen information, pulse rate information, and respiratory rate information can be transmitted to the external device 40 on the backend.

Continuing from the previous steps, the second notification signal can alert the backend personnel to pay attention to the user's sleep condition. If the backend personnel, upon reviewing the blood oxygen information, pulse rate information, and respiratory rate information, determine that the user needs to be awakened, they can issue a warning notification to the communication unit 34. The communication unit 34 then transmits the warning notification to the processing unit 22, enabling the processing unit 22 to control the alert unit 26 to issue a warning to awaken the user. At the same time, the external device 40 can also store this information, such as the time of receiving the message and the blood oxygen information, pulse rate information, and respiratory rate information detected at that moment. This data can be recorded to track the user's sleep status and provide reference for subsequent treatment.

Besides, whether the human body is experiencing hypoxia can also be determined through blood oxygen levels. In this embodiment, the processing unit 22 can directly generate a warning signal to the alert unit 26 when the blood oxygen information is below the third blood oxygen threshold. The alert unit 26 then issues a warning based on the warning signal to awaken the user. In this embodiment, the third blood oxygen threshold is set at 90 mm Hg. If the blood oxygen falls below this threshold of 90 mm Hg, it indicates that the user may be experiencing hypoxia due to respiratory cessation. In this situation, the processing unit 22 directly generates a warning signal to the alert unit 26. The alert unit 26, based on the warning signal, issues an audible warning to awaken the user.

In summary, the present invention, when installed in the ear canal, can effectively detect and record the user's physiological state. It provides comfort during use and is easy to carry. When installed, it closely adheres to the ear canal, providing a dark environment that enhances the detection conditions for the pulse oximetry unit and improves signal stability. Importantly, the present invention can timely awaken the user when sleep apnea occurs, and it elevates the alert level when the user adopts a sleeping position with a higher probability of experiencing sleep apnea. Additionally, the present invention can transmit physiological information to a remote backend in real-time, allowing backend personnel to monitor the user's physiological condition at any time and record the physiological state. This can be effectively applied to subsequent treatment.

Many details are often found in the relevant art and many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. A sleep state monitoring earplug, wearable in an ear canal of a user, the sleep state monitoring earplug comprising:
a main body; and a detection module inside the main body, the detection module comprising:
a processing unit;
a respiratory detection unit connected to the processing unit, and configured to generate respiratory rate information to the processing unit; and
an alert unit connected to the processing unit and configured to generate a warning signal if the respiratory rate information is lower than a respiratory threshold, and the alert unit to issue a warning according to the warning signal.

2. The sleep state monitoring earplug of claim 1, wherein the detection module comprising:
a pulse oximetry detection unit connected to the processing unit, and configured to generate blood oxygen information and pulse rate information to the processing unit; and
a communication unit connected to the processing unit and an external device, the communication unit being controlled by the processing unit and configured to transmit the blood oxygen information, the pulse rate information and the respiratory rate information to the external device.

3. The sleep state monitoring earplug of claim 2, wherein the processing unit is further configure to generate a first notification signal to the communication unit if the processing unit determined that the blood oxygen information is below a first blood oxygen threshold, the pulse rate information is outside a first pulse rate range, or the respiratory rate information is outside a first respiratory rate range, and the communication unit transmits the first notification signal to the external device.

4. The sleep state monitoring earplug of claim 3, wherein the detection module further comprising:
an orientation detection unit connected to the processing unit, and configured to generate angle information to the processing unit, the processing unit generates a second notification signal to the communication unit if the processing unit determines that the angle information falls within a specified range, and the blood oxygen information is below a second blood oxygen threshold, the pulse rate information is outside a second pulse rate range, or the respiratory rate information is outside a second respiratory rate range, and the communication unit transmits the second notification signal to the external device.

5. The sleep state monitoring earplug of claim 4, wherein the orientation detection unit is a gyroscope.

6. The sleep state monitoring earplug of claim 4, wherein the first blood oxygen threshold is higher than the second blood oxygen threshold, the second pulse range is narrower than the first pulse range, and the second breathing range is narrower than the first breathing range.

7. The sleep state monitoring earplug of claim 2, wherein the processing unit further generates the warning signal to the alert unit if the blood oxygen information is determined to be lower than the third blood oxygen threshold, causing the alert unit to issue the warning according to the warning signal.

8. The sleep state monitoring earplug of claim 1, wherein the alert unit is a sound unit.

9. The sleep state monitoring earplug of claim 1, wherein the respiratory detection unit is a microphone.

10. The sleep state monitoring earplug of claim 1 further comprising:
a battery module connected to the processing unit to supply power.
